(19) United States
(12) Patent Application Publication
(10) Pub. No.: US 2006/0194746 A1
(43) Pub. Date: Aug. 31, 2006

(54) Method and composition to inhibit infections with Helicobacter pylori by intake of procyanidins from type B and C

(76) Inventor: Peter Rohdewald, Munster (DE)

Correspondence Address: Eric Sinn, Esq.;Mintz, Levin, Cohn, Ferris, Glovsky & Popeo, P.C., The Chrysler Center, 666 Third Avenue, 24th Floor, New York, NY, 10017, US

(21) Appl. No.: 11/069,523

(22) Filed: Feb. 28, 2005

(51) Int. Cl. A61K 31/7048; A61K 31/353

(52) U.S. Cl. 514/27; 514/456

(57) ABSTRACT

Methods and compositions, including, foods and beverages, for treating, preventing or ameliorating Helicobacter pylori-associated disorders, particularly disorders of the gastrointestinal tract such as peptic ulcers of the stomach are provided by this disclosure. In one preferred aspect, the method comprises administering to a Helicobacter pylori infected subject a therapeutically effective amount of a composition comprising Type-B procyanidins, Type-C procyanidins, or a mixture thereof.

### TECHNICAL FIELD

The invention relates to methods, pharmaceutical compositions, foods and beverages for treating, preventing or ameliorating Helicobacter pylori-associated disorders, particularly disorders of the gastrointestinal tract such as peptic ulcers of the stomach.

### BACKGROUND

H. pylori (formerly called Campylobacter pylori) is a gram-negative bacterium that can colonize the human gastric mucosa. This bacterium was frequently detected in stomach biopsy specimens from patients with gastritis or gastric ulcers. Further, epidemiological studies have indicated that this bacterium is the causative agent for gastritis, gastric ulcers, and duodenal ulcers and is associated with disorders such as gastric cancer.

Once H. pylori colonizes gastric mucosa, it survives and persists in the stomach and cannot be eradicated without medical intervention. Colonization of the stomach by Helicobacter pylori is followed by chronic gastritis type B. Eighty-five percent of cases of chronic gastritis are caused by Helicobacter pylori infections. Unless H. pylori is completely eliminated from the stomach by antibiotic therapy, the infection will return to the same level as before treatment within about a month after cessation of antibiotics administration.

Antibiotic treatment is complicated by the fact that the low pH environment of the stomach tends to inactivate many antiboitics. For this reason, antibiotics are commonly administered with medicine (e.g., proton pump inhibitor) which suppresses the secretion of gastric acid. However, the prolonged administration of antibiotics and proton pump inhibitors results in undesirable side effects for the patient and increased antibiotic-resistant strains in the environment. Furthermore, while H. pylori induced gastritis may not present symptoms, chronic infection with Helicobacter pylori can cause gastric cancer.

A large percentage of patients remain without any treatment for Helicobacter pylori despite ulcer formation because despite ulcer formation, chronic gastritis is not always diagnosed. In some cases, patients present mild symptoms or are asymptomatic.

At present it is believed that eradication of H. pylori from the stomach is essential for fully treating peptic ulcers and for the prevention of stomach cancer. There is a longfelt but unmet need to prevent and to treat infections with Helicobacter pylori on the basis of a natural supplement rather than systemic antibiotic(s) and/or proton pump inhibitors.

### BRIEF DESCRIPTION OF THE INVENTION

The present invention relates to methods and compositions, including pharmaceutical compositions, for the treatment and prevention of H. pylori-associated disorders. It is an object of the present invention to provide an effective and safe inhibitor of H. pylori colonization which is associated with the occurrence of peptic ulcers without the disadvantages of side effects associated with current methods of antibiotic and proton pump inhibitor administration. Another object of the invention is to provide a medicament and food useful for treating or preventing peptic ulcers. The methods and compositions of the invention may be used for the treatment and/or prevention of bacteria-associated gastrointestinal disorders such as H. pylori-associated gastrointestinal disorders.

One embodiment of the invention is directed to a method for treating, ameliorating or inhibiting Helicobacter pylori infection in mammals as described in detail herein. The method involves the step of administering to a mammal a composition comprising procyanidins (e.g., B-type or C-type procyanidins) in an effective amount for inhibiting Helicobacter pylori colonization. While any mammal may be treated by the method of the invention, a preferred animal for treatment is a human or commercially valuable animals and livestocks.

Administration may be topical, oral or parental. Thus, the procyanidins formulation may comprise pharmaceutical formulation for oral application, for injection, or for topical application.

In a preferred embodiment, the composition comprise B-type procyanidins with less than 50% of the total procyanidins being of the A-Type. In a more preferred embodiment, less than 30% of the total procyanidins are the A-Type. In an even more preferred embodiment, less than 10% of the total procyanidins are of the A-Type. In the most preferred embodiment, the composition is comprised mostly of non-A-Type procyanidins and is substantially free of A-Type procyanidins. Non-A Type procyanidins includes, for example, B-type procyanidins, C-Type procyanidins, or a mixture of B-type and C-type procyanidins. The composition may contain the above mentioned procyanidins (B-type procyanidins, C-type procyanidins, or a mixture of both) in the range of 10% to 100% by weight. For example, the composition may contain B-type (or B-type and C-type) procyanidins at a concentration of at least 10%, at least 25%, at least 50%, at least 75%, at least 90%, at least 95% or at least 98%.

The procyanidins compositions for use in the methods of the invention may be obtained from plant materials or by synthesis. The procyanidins containing composition may be administered in an amount of between 10 mg to 6 g per day per person (e.g., a human of 50 kg body weight to 70 kg body weight such as, for example, 50 kg, 55 kg, 60 kg, 65 kg or 70 kg body weight). For example, the procyanidins composition may be incorporated into a food for human or animal consumption. The food may be solid, or liquid (such as a beverage/drink). The procyanidins containing food may be consumed to provide a mammal with 10 mg to 6 g per day per person (e.g., a human of 50 kg body weight or 70 kg body weight). In one preferred embodiment, the procyanidins composition may be Pycnogenol®.

The methods and compositions of the invention may be used to treat or prevent diseases associated with Helicobacter pylori infection, such as, for example, Helicobacter pylori-associated gastrointestinal disease. Examples of gastrointestinal disease treatable by the methods and compositions of the invention includes gastric peptic ulcer, duodenal peptic ulcer, gastritis, duodenitis, non-ulcer dyspepsia and gastric carcinoma. The procyanidins compositions of the invention may be bacteriocidal, bacteriostatic, or may reduce the adhesion of bacteria (Helicobacter pylori) to stomach epithelial.

The term “treating” in its various grammatical forms in relation to the present invention refers to preventing, curing, reversing, attenuating, alleviating, ameliorating, inhibiting minimizing, suppressing, or halting (1) the deleterious effects of a disorder associated with Helicobacter pylori infection, (2) disorder progression, or (3) disorder causative agent (e.g., Helicobacter pylori). Treating also includes inhibiting Helicobacter pylori attachment (adhesion) to a body surface such as a gastrointestinal surface. The gastrointestinal surface includes, at least, the lining, interior surface, or epithelial of the stomach or intestine.

Other objects and advantages as well as the nature of the present invention will be apparent from the following description.

### BRIEF DESCRIPTION OF THE FIGURES

FIG. 1 depicts B1 procyanidin dimers identified in Pycnogenol®.

FIG. 2 depicts B3 procyanidin dimers identified in Pycnogenol®.

FIG. 3 depicts B6 procyanidin dimers identified in Pycnogenol®.

FIG. 4 depicts B7 procyanidin dimers identified in Pycnogenol®.

FIG. 5 depicts a procyanidin trimer identified in Pycnogenol®.

FIG. 6 depicts chemical structures of A- B- and C-type Procyanidins®.

FIG. 7 (a) shows the decline of growth of Helicobacter pylori in percentage relative to control. (b) shows that Helicobacter pylori growth was inhibited significantly six hours after administration of Pycnogenol®. (c) shows that Helicobacter pylori growth was completely inhibited 24 hours after administration of Pycnogenol®. (d) shows that Pycnogenol® inhibits adherence of H. pylori to gastric epithelial cells in a dose dependent manner.

### DETAILED DESCRIPTION OF THE INVENTION

Proanthocyanidins designates a group of flavanoids that includes the subgroups procyanidins, prodelphinidins and propelargonidins. While the procyanidins of this disclosure are not limited to those derived from any source, one preferred source of procyanidins is French maritime pine bark extract (Pycnogenol®).

Helicobacter pylori (H. pylori) and Campylobacter pylori (C. pylori) are understood to refer to the same organism. Hence, these terms are interchangeable.

Pinus pinaster (P. pinaster) and Pinus maritima (P. maritime), are understood to refer to the same organism. Hence, these terms are interchangeable.

One aspect of the invention relates to a pharmaceutical composition for the treatment and/or prevention of H. pylori-associated disorders comprising as active ingredient a therapeutically effective amount of procyanidins compound which inhibits the growth and/or adhesion of H. pylori.

The pharmaceutical composition of the invention may be particularly suitable for the treatment and/or prevention of H. pylori-associated gastrointestinal disorders, specifically, H. pylori-associated gastric and/or duodenal peptic diseases. In addition the compositions of the invention may be used for the treatment of gastritis, duodenitis, non-ulcer dyspepsia, and for the prevention of gastric carcinoma.

The active ingredient in the pharmaceutical compositions of the invention is preferably a compound which has bacteriostatic (bacteria inhibiting) activity or bactericidal (bacteria killing) activity towards H. pylori.

In preferred embodiments, the active compound comprised in the pharmaceutical composition of the invention is a composition comprising a source of procyanidins.

We have found that proanthocyanidins are capable of blocking the colonization of the stomach by Helicobacter pylori and to inhibit its growth. It is possible that proanthocyanidins inhibit H. pylori growth by binding bacterial walls proteins and hinder mobility and metabolism. Antibacterial activity was reported for proanthocyanidins isolated from Oboubaka aubrevelli, Krameria lappacea, Machaerium floribundum, Tormentilla erecta (Scalbert, 1991, Phytochemistry, 30: 3875-3883). Procyanidins from apples inhibit the growth of Streptococcus mutans (Yamagida et al. 2000, J. Agric. Food Chem. 48: 5666-5671).

A series of proanthocyanidins tested showed moderate activity against several bacteria and yeasts (Kolodziej et al., 1999, Planta Med., 65(5): 444-446). A special configuration of proanthocyanidins, with A-Type linkages, isolated from cranberries, inhibited adhesion of Escherichia coli to the urogenital epithelium, (Foo et al. 2000, Phytochemistry. 54(2): 173-181; Foo et al. 2000, J. Nat. Prod., 2000, 63(9): 1225-1228) adhesion of Helicobacter pylori to stomach cells (Burger et al., 2000, 29(4): 295-301) and adhesion of oral bacteria to teeth (Weiss et al., 10999. J. Amer. Dental Assoc., 129: 1719).

B-type procyanidins were reported to be inactive in inhibiting adhesion of Echerichia coli to epithelial cells (Foo et al. 2000, J. Nat. Prod., 2000. 63(9): 1225-1228). Surprisingly, we found in the present invention that B-type procyanidins are able to inhibit dose-dependently the adhesion of Helicobacter pylori to gastric cells. Furthermore, a bacteriostatic effect of procyanidins from B-type could be demonstrated.

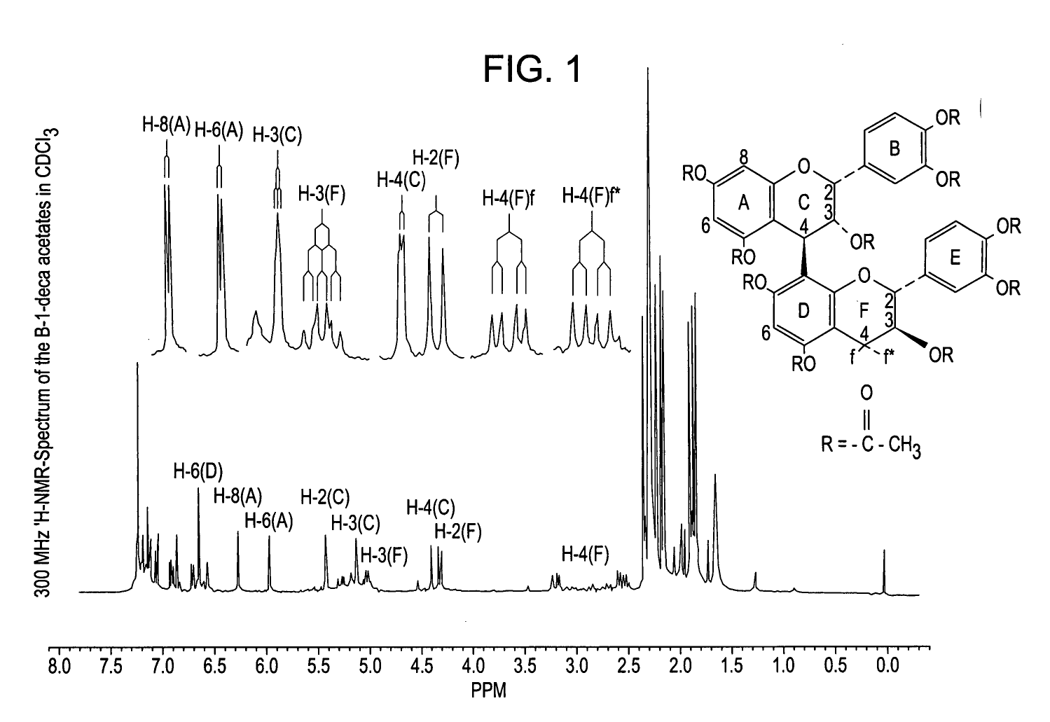

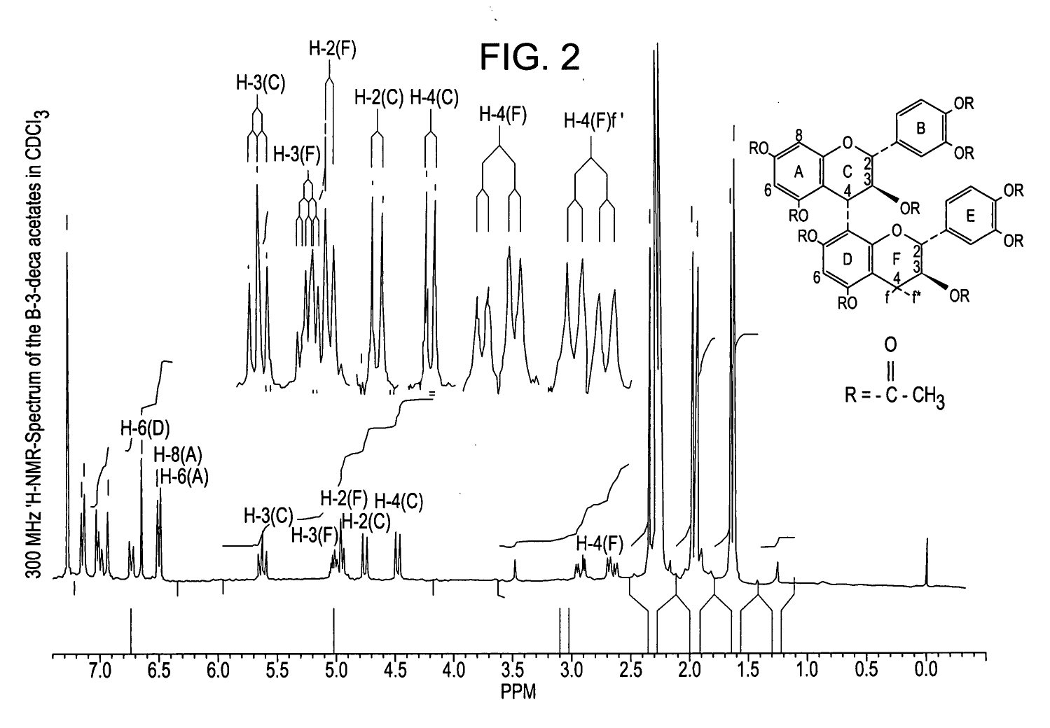

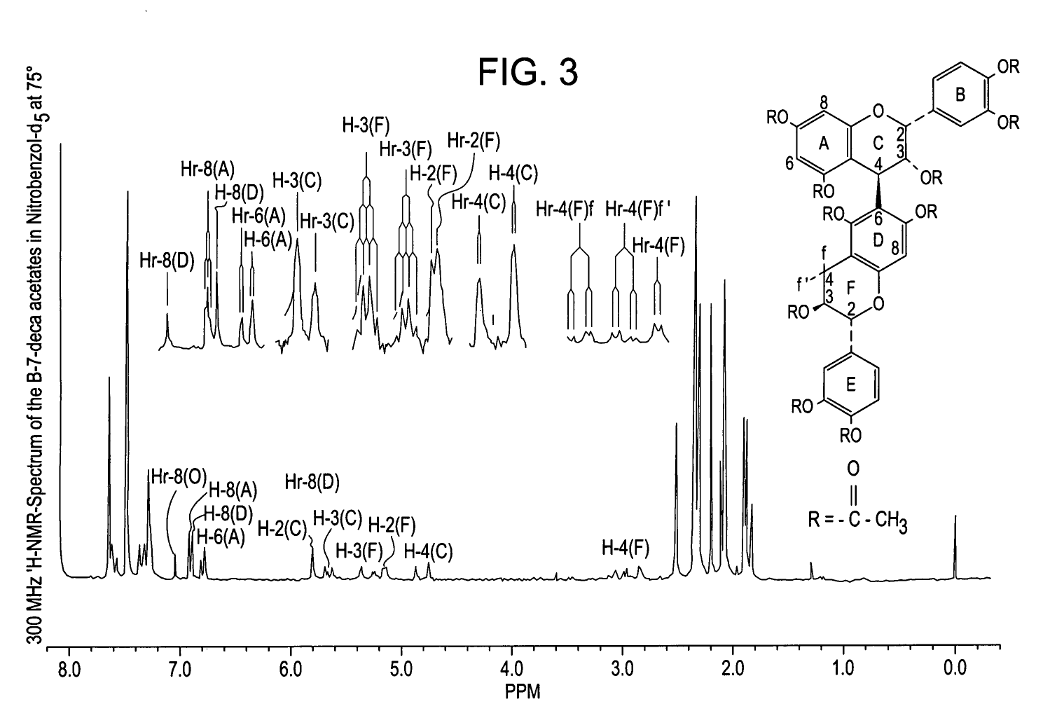

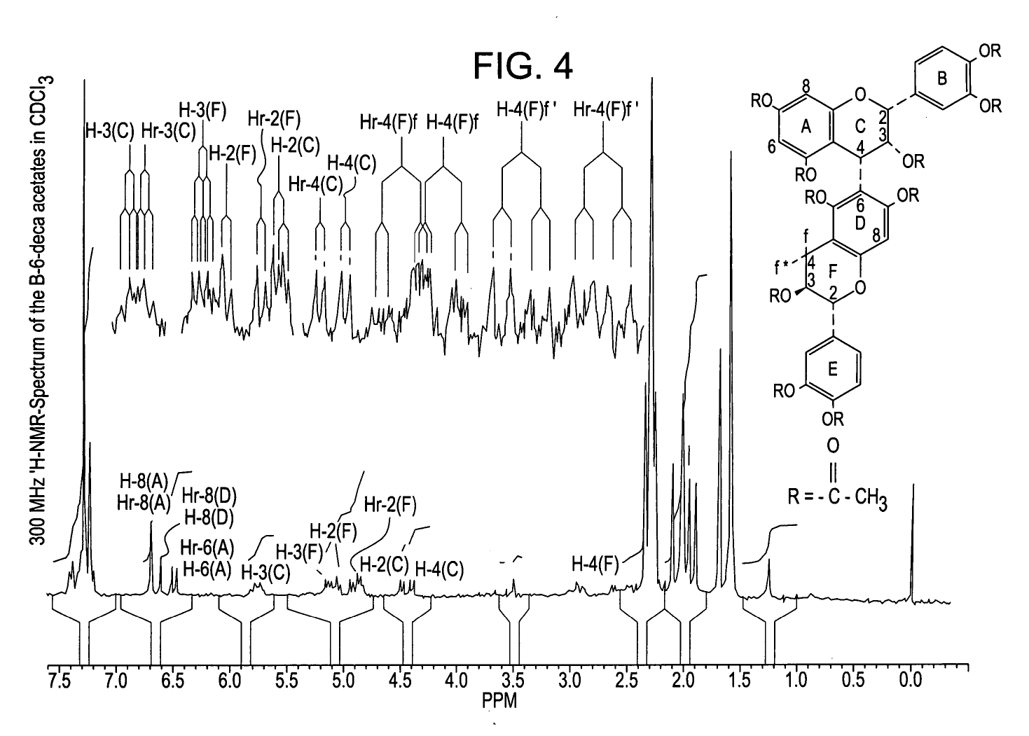

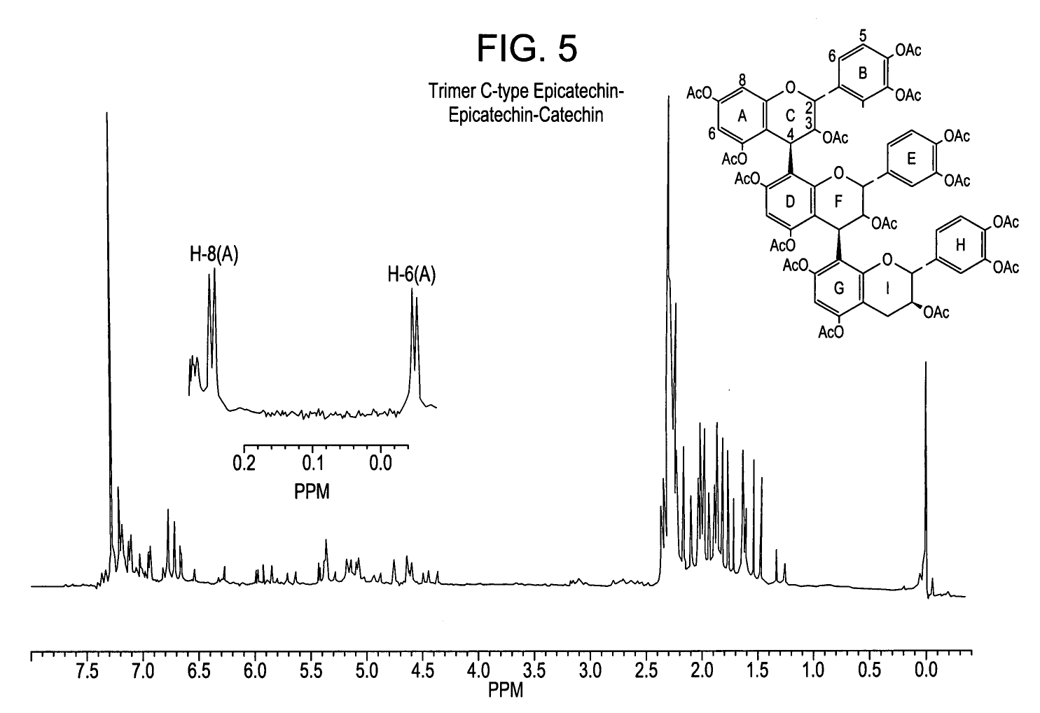

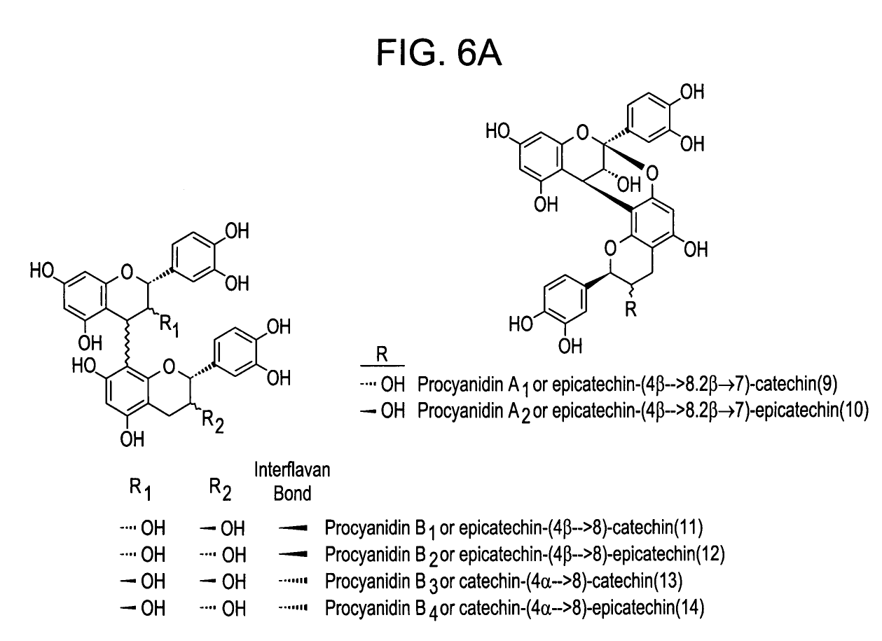

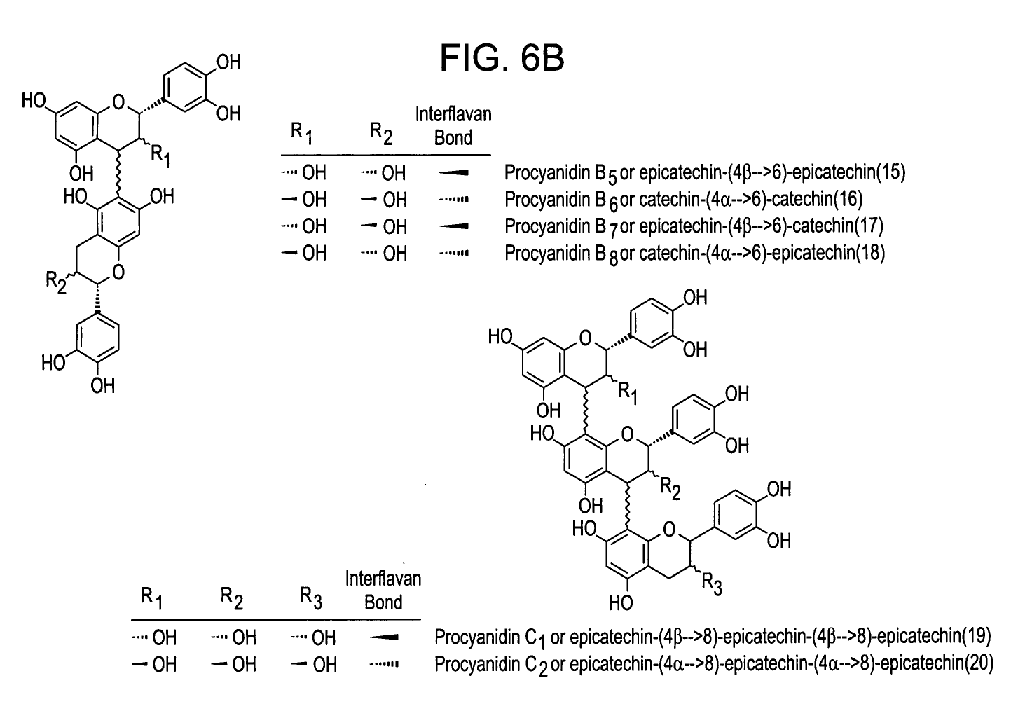

The procyanidins used to inhibit adhesion and growth of Helicobacter pylori were extracted from the bark of Pinus pinaster Ait. That extract, marketed by Horphag Research under the trade name Pycnogenol®, consists mainly of procyanidins (65-75%). These procyanidins, biopolymers formed from catechin and epicatechin subunits, belong to the type B procyanidins. (Thesis Pirasteh, Münster, 1988). Four different dimers, B1, B3, B6 and B7 could be identified using a combination of NMR and MS spectroscopic methods (FIG. 1-4). The same type of binding can be found in higher oligomers, as for example in the trimer isolated from pine bark extract (FIG. 5) (Thesis Pirasteh, Münster, 1988). These procyanidins differ from the A-type procyanidins fundamentally by the fact that in the A-type procyanidins two monomers are linked by two bonds to each other, whereas in the B-type procyanidins only one single bond links the two flavanyl units (FIG. 6).

These different structures influence the configuration of the molecules. Whereas in the B-type dimers and C-type oligomers the flavanyl units are free to rotate around the C—C bond, the configuration of the A-dimer is fixed by the double linkage C—C and the ether-bridge, the C—O—C bond. Such a different structure influences physicochemical parameters of the procyanidins as well as their biological activities. The following examples demonstrate, unexpected in context with the reports from Foo et al. (Foo et al. 2000, J. Nat. Prod., 2000, 63(9): 1225-1228) that procyanidins from B-type, concentrated in an extract from the French maritime pine (Pycnogenol®), are able to reduce the adherence of Helicobacter pylori to gastric epithelial cells and reduce the infection rate of these cells and to exhibit a bacteriostatic action on H. pylori.

B-type procyanidins from B-type are contained in a wide range of plants as for example in pine barks, grape seeds, cacao beans, cola nuts, apples. Procyanidins from B-type may be extracted from these sources of natural procyanidins using polar solvents, preferably water and ethanol, by usual extraction methods including supercritical fluid extraction. The extracts are purified and concentrated by ultra filtration, reverse osmosis, fractionated precipitation or other methods. The purified solutions are spray dried or freeze dried to obtain concentrated type-B procyanidins extracts. The extracts present the raw material for production of powder, capsules, tablets, solutions and technologically improved dosage forms as microcapsules, sustained release formulations, micro emulsions and other forms directed to a prolonged residence time of the procyanidins inside the stomach. If necessary, B-type procyanidins, C-type procyanidins, or a mixture of both B-type and C-type may be purified from an extract containing, for example, a mixture of A- B- and C-type procyanidins, using conventional purification techniques such as column chromatography. That is, chromatography may be performed on a plant extract to provide a pure form of B-type, C-type, or mixture of B-type and C-type procyanidins which has reduced (or complete absence of) A-type procyanidins.

All references, patents, and patent applications cited in this disclosure are incorporated by reference in their entirety.

### EXAMPLES

### Example 1

### Inhibition of Growth of Helicobacter Pylori by Procyanidins

Three strains of Helicobacter pylori were cultivated in Brain Heart Infusion Broth Medium (BHI) at 37° C. 1.5×10⁷ H. pylori (optical density OD=0.1) were incubated in RPMI medium plus 10% fetal calve serum for 48 hours with Pycnogenol® in concentrations of 12.5, 25, 50, 125, 250, 500 and 1000 μg/ml. After 48 hrs, concentrations of Helicobacter pylori were controlled by measuring optical density. Untreated controls were grown under the same conditions.

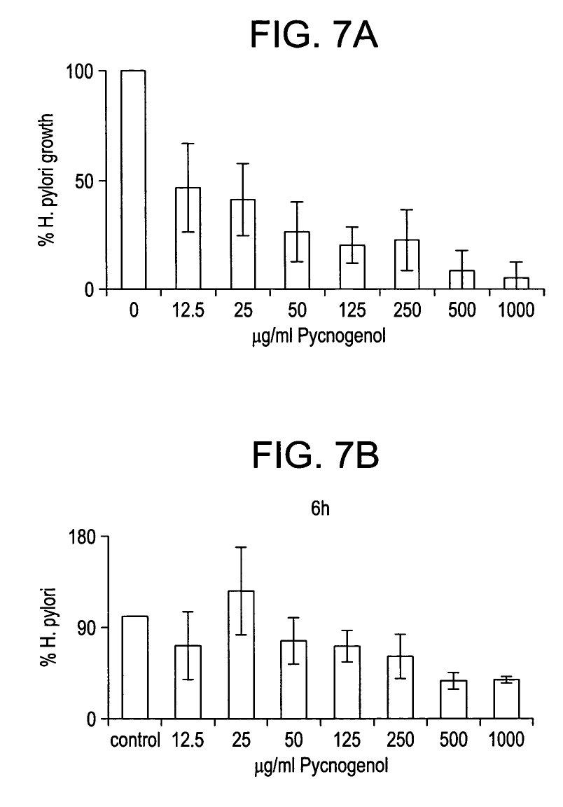

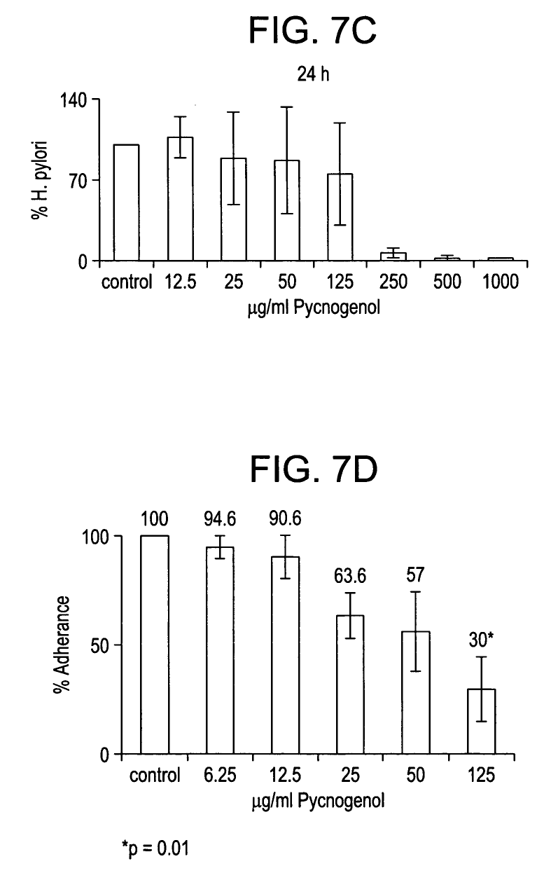

FIG. 7a shows the decline of growth of Helicobacter pylori in the treated samples (in percentage) relative to controls. A 90% inhibition of growth was achieved with 500 μg/ml Pycnogenol® for all strains.

### Example 2

### Inhibition of Growth of Helicobacter Pylon in Infected Gastric Cells by Procyanidins

Gastric cells were cultivated in RPMI medium plus 10% fetal calve serum for 24 hrs at 37° C. After changing the cell culture medium with RPMI medium plus 10% fetal calve serum plus 20% BHI medium, cells were infected with 10⁵ H. pylori. Three hours after infection, cells were incubated with Pycnogenol® in concentrations from 12.5 to 1000 μg/ml. Helicobacter pylori concentration in cells was determined after 6 and 24 hrs using the urease test after adding urea to the cell culture. Six hours following infection, 500 and 1000 μg/ml Pycnogenol® inhibited growth significantly (FIG. 7b). Twenty-four hours after addition of Pycnogenol®, complete inhibition of growth of Helicobacter pylori was found at concentrations exceeding 250 μg/ml (FIG. 7c).

### Example 3

### Inhibition of Adherence of Helicobacter Pylori to Stomach Epithelial Cells by Procyanidins

Stomach epithelial cells (AGS-cells) were incubated using culture conditions as described before with Helicobacter pylori in presence of Pycnogenol® in concentrations from 6.25 to 125 μg/ml. Three hours after incubation cells were washed with medium to remove non-adherent Helicobacter pylori and Pycnogenol®. Concentrations of Helicobacter pylori on cells was determined with the urease-test (described, for example, in Kuo CH et al., Hepatogastroenterology. 49(47):1191-4 or commercially available from Kimberly Clark as CLOtest® (Roswell, Ga., USA)). As demonstrated by FIG. 7d, Pycnogenol® inhibited dose-dependently adherence of H. pylori to gastric epithelial cells. In presence of 125 μg/ml Pycnogenol®, only 30% of the population of Helicobacter pylori were adherent to the gastric cells.

We claim:

1. A method for treating, preventing, ameliorating or inhibiting Helicobacter pylori infection in mammals comprising the step of administering to said mammal a procyanidins composition comprising B-type procyanidins in an effective amount for inhibiting Helicobacter pylori colonization.

2. The method of claim 1 wherein said mammal is a human.

3. The method of claim 1 wherein said administration is oral.

4. The method of claim 1 wherein said composition comprises less than 10% A-type procyanidins.

5. The method of claim 1 wherein said composition is substantially free of A-type procyanidins.

6. The method of claim 5 wherein the procyanidins are type-B or higher procyanidins dimers without type A bonds.

7. The method of claim 6 wherein said procyanidins are isolated from plant material or obtained by synthesis.

8. The method of claim 1 wherein the procyanidins are at a concentration in the composition of between 10% to 100% of total weight.

9. The method of claim 1 in wherein the composition is a pharmaceutical formulation for oral administration.

10. The method of claim 1 in which the procyanidins composition is administered in an amount of between 10 mg to 6 g per day per person.

11. The method of claim 1 in which the procyanidins composition is incorporated into a food.

12. The method of claim 11 wherein the food is administered to provide 10 mg to 6 g per day per person of said procyanidins composition.

13. The method of claim 1 wherein said Helicobacter pylori infection is associated with a Helicobacter pylori-associated gastrointestinal disease.

14. The method of claim 13 wherein said gastrointestinal disease is selected from the group consisting of gastric peptic ulcer, duodenal peptic ulcer, gastritis, duodenitis, non-ulcer dyspepsia and gastric carcinoma.

15. The method of claim 1 wherein said method reduces the adhesion of Helicobacter pylori to stomach epithelial cells.

16. A composition for inhibiting Helicobacter pylori infection in mammals comprising a procyanidins composition which comprises less than 10% A-Type procyanidins.

17. The composition of claim 16 wherein said composition comprises less than 5% A-type procyanidins.

18. The composition of claim 16 wherein said composition is essentially free of A-type procyanidins.

19. The composition of claim 16 wherein said composition comprises B-Type procyanidins and C-Type procyanidins.
